(19) United States
(12) Patent Application Publication
(10) Pub. No.: US 2025/0331828 A1
(43) Pub. Date: Oct. 30, 2025

(54) METHOD, DEVICE AND SYSTEM FOR OVARIAN CANCER DETECTION

(71) Applicant: Emmanuel MASTORAKIS, Monte-Carlo (MC)

(72) Inventor: Emmanuel MASTORAKIS, Monte-Carlo (MC)

(21) Appl. No.: 19/144,734

(22) PCT Filed: Oct. 31, 2023
(86) PCT No.: PCT/IB2023/060964

§ 371 Date: Jun. 30, 2025

Related U.S. Application Data
(60) Provisional application No. 63/381,638, filed on Oct. 31, 2022.

(51) Int. Cl. A61B 10/00

(52) U.S. Cl. CPC A61B 10/0045; A61B 2010/0074

(57) ABSTRACT

A uterine lavage kit for collecting a solution of uterine cells of a female subject, including a body, an insertion cannula, a syringe for injecting a solution via the insertion cannula into the uterine cavity, a collection cup for collecting the uterine cells solution egressed from the uterine cavity after the injection, a removable container, and an extraction cannula in fluidic connection with the collection cup, the extraction cannula configured to guide the solution from the collection cup towards the removable container.

### BACKGROUND

Endometrial and ovarian cancers are the sixth and seventh most common cancers in women worldwide. Ovarian cancer remains one of the most lethal cancers for females. Endometrial cancer is the most common gynecologic malignancy, and the incidence and associated mortality are increasing. Ovarian cancer is also the most lethal reproductive tract malignancy. Despite the immediate need to detect these cancers at an earlier stage, there are no effective screening methodologies or protocols. It has been estimated by the United States National Cancer Institute that in 2017 approximately 60,000 women were diagnosed with endometrial cancer, also known as uterine cancer, and 22,400 women with ovarian cancer. The most disturbing evidence is that last year close to 25,000 women succumbed to these two diseases.

Uterus flushing processes are well-known like for example saline infusion sono-hysterography (SIS or SHG) which is employed to evaluate the uterine cavity prior to commencement of assisted conception. Intra-uterine lesions play an important role in the outcome of assisted conception procedures. Uterine flushing with saline solution is also used as a treatment for unexplained infertility. Saline infusion sono-hysterography (SHG) is a procedure to evaluate the uterus and the shape of the uterine cavity. SHG uses ultrasound and sterile fluid to show the uterus and endometrial (uterine lining) cavity. The ovaries are also seen at the time of SHG. The purpose is to detect any abnormalities. The current gold-standard for diagnosing endometrial cancer requires evaluation under general anesthesia in the operating room by a surgeon. Currently, there are no clinically proven screening tests or in-patient procedures that can be used in place of operating room-based hysteroscopy.

There are currently no recommended screening tests for ovarian cancer.

For example, the preliminary test without any anesthesia that can be done in the doctor's office using the Pipelle de Cornier™, also called Pipelle endometrial suction curette, often leads to false-negative, due to the difficulty to ensure a complete lavage of the uterine cavity. Therefore, even if this test is negative, the doctor normally recommends an invasive hysteroscopy, requiring at least partial anesthesia, to confirm the diagnostic. The anesthesia can be hard on the patient, long and costly, requiring time at the hospital.

With respect to existing uterine sealing systems, the state of the art proposed inflatable balloons or expanded foams. These systems make it possible to seal the bottom of the cervix and then provide by a cannula a fluid in the uterus by passing through the seal. A disadvantage is that this principle of tightness of the uterus can hurt and be painful for the patient and require anesthesia which is not desired. Another background solution, the Speiser Catheter that has been offered by the company OncoLab Diagnostics GmbH of performing the Lavage for Ovarian Cancer (LOC) test. This device requires local anesthesia, and during the lavage, sterile saline is slowly flushed into the uterine cavity by a syringe through one line of a three-way catheter. Simultaneously, the fluid is aspirated with a second syringe through the other line. The catheter is fixed in the uterine cavity by a small inflatable balloon. The lavage sample is mixed with a stabilizer fluid in a third syringe and shipped to a laboratory for testing. In sum, this solution is complex, requires the handling of three different syringes, and also requires local anesthesia.

In light of the above described numerous disadvantages and problems of the current state of the art for systems, devices, and methods for the early detection of endometrial or ovarian cancer, novel and improved solutions are strongly desired.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIGS. 1A-1E are different views of a disposable single-use uterine lavage device 100, in accordance with some embodiments.

FIG. 2A is a side perspective view of a disposable uterine lavage device as a kit, in accordance with some embodiments.

FIG. 2B is a side perspective view of elements packaged inside blister packaging, in accordance with some embodiments.

FIG. 3A is a side perspective close-up view of a body, a container, and a syringe of the kit, in accordance with some embodiments.

FIG. 3B is a view of two cups of the kit, in accordance with some embodiments.

FIG. 3C is a side view of the elements of the kit where a cup is placed over an extraction cannula, in accordance with some embodiments.

FIG. 4 is a schematic flowchart of a method of assembling and using the disposable uterine lavage device, in accordance with some embodiments.

FIGS. 5A and 5B are side cross-sectional views of a cup with and without a cannula inserted, in accordance with some embodiments.

FIGS. 6A and 6B are side cross-sectional views of the syringe, in accordance with some embodiments.

FIGS. 7A-7G are perspective views of different elements of the spraying or outlet tip, in accordance with some embodiments.

FIGS. 8A-8I and 8K-8O are side and perspective views of different stages in a method of unpacking, assembling, and using the disposable uterine lavage device, in accordance with some embodiments.

FIGS. 9A-9F are views of steps of operation of a safety mechanism, in accordance with some embodiments.

Herein, identical reference numerals are used, where possible, to designate identical elements that are common to the figures. Also, the images are simplified for illustration purposes and may not be depicted to scale.

### DETAILED DESCRIPTION

The following disclosure provides many different embodiments, or examples, for implementing different features of the provided subject matter. Specific examples of components, values, operations, materials, arrangements, or the like, are described below to simplify the present disclosure. These are, of course, merely examples and are not intended to be limiting. Other components, values, operations, materials, arrangements, or the like, are contemplated. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

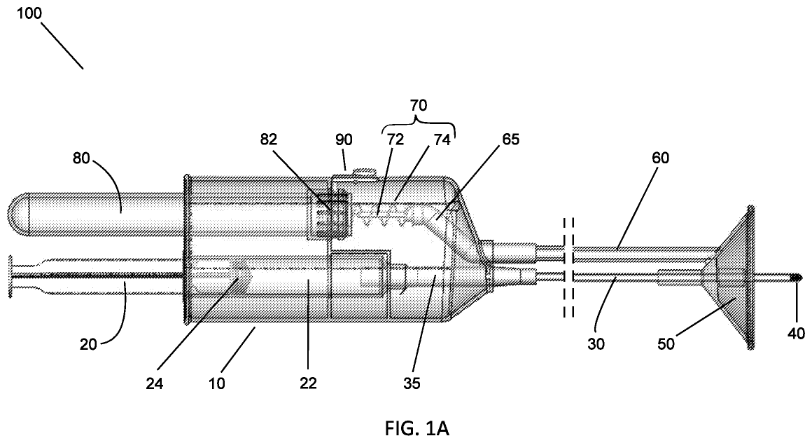

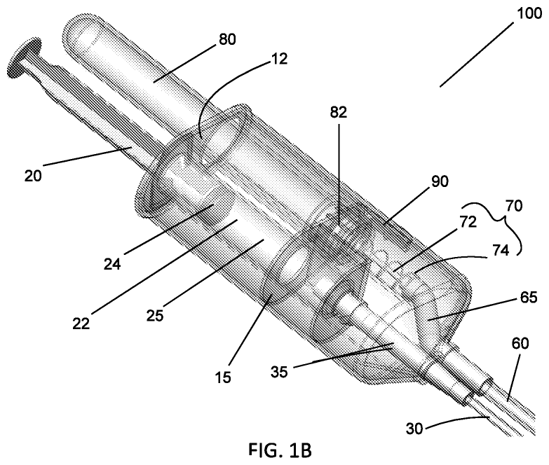

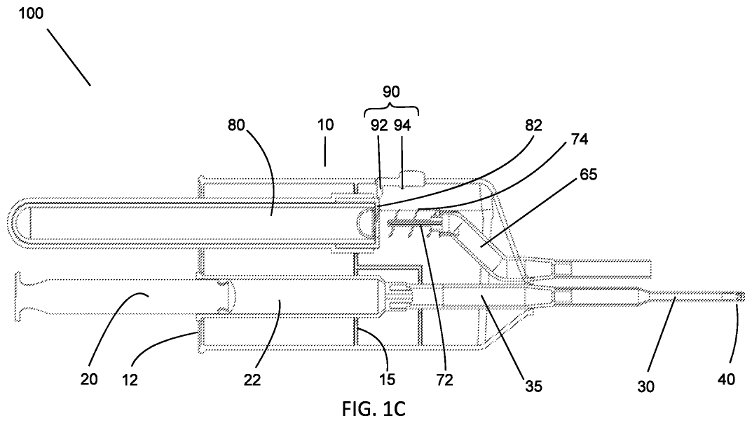

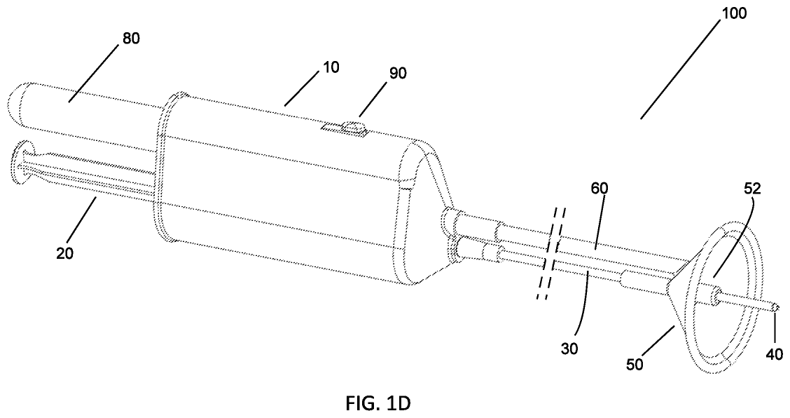

FIGS. 1A to 1E show different views of a disposable single-use uterine lavage device 100 according to a first aspect of the present invention, with FIG. 1A showing a side view, FIG. 1B showing a side perspective view with transparent elements, FIG. 1C showing a cross-sectional view, FIG. 1D showing a side perspective view with non-transparent elements with an inserted cup 50, and FIG. 1E showing a cross-sectional view with an inserted cup 50, device 100 including a body 10 for holding several elements of the device 100 together and for allowing a user to hold the device 100 with a single hand, a prefilled syringe 20 for containing and then spraying or injecting a solution into the uterus, syringe 20 removably attached to the body 10, an insertion cannula 30 for guiding the solution ejected from syringe 20 through body 10 via fluidic pathway 35, a spraying tip 40 for spraying and dispensing the solution inside the uterus, a cup 50 for collecting the lavage solution released by the uterus by gravity, an extraction cannula 60 for collecting the solution from cup 50 to guide it towards body 10, a needle and spring system 70 for engaging with a container 80, and a safety mechanism 90 for allowing the needle 72 of needle and spring system 70 to pierce or otherwise penetrate a seal 82 of container 80. Body 10 is shown to made of transparent material such that a scale 22 of syringe can be seen by the user through the transparent walls of body 10, to show the interior of body 10, and allows of a visible inspection of a connection between needle 72 and container 80.

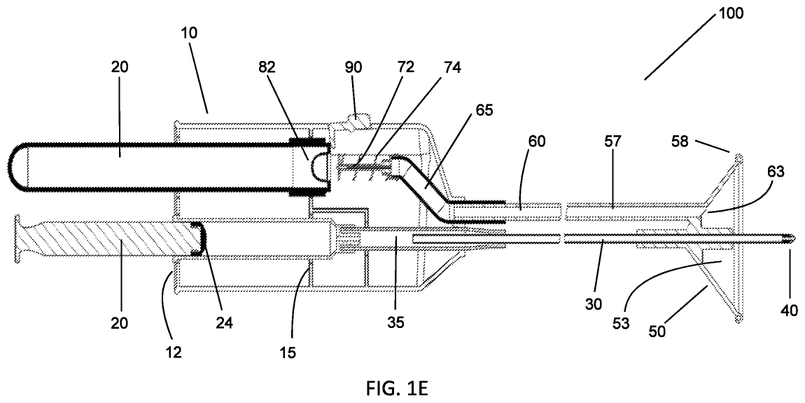

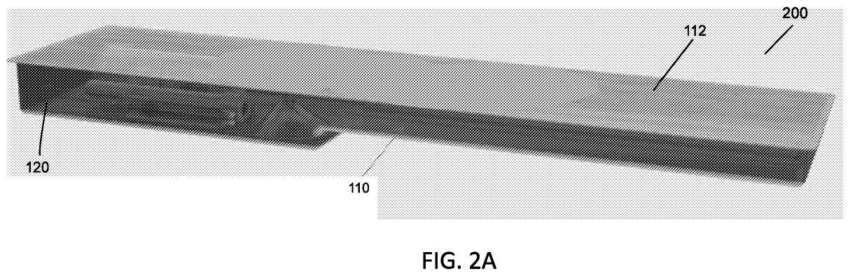

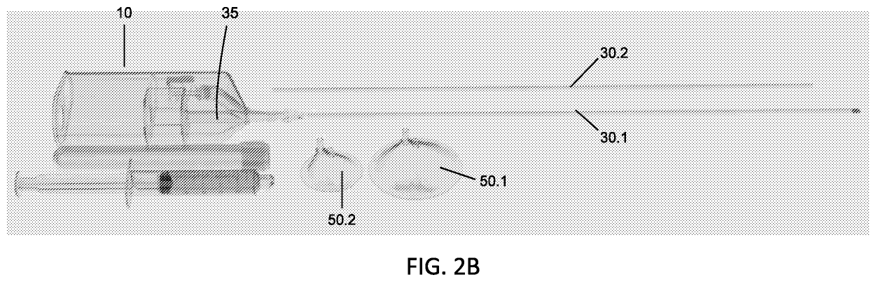

FIG. 2A shows a side perspective view of the newly manufactured and sterilized disposable uterine lavage device 100 as a kit 200 packaged into a sterile protective blister packaging 110 for single use, or another type of sterile packaging, according to a second aspect of the present invention.

FIG. 2B shows a side perspective view of the elements packaged inside the blister packaging 110 that can be assembled to be an operational disposable uterine lavage device 100, shown at a position where the elements are packaged inside a sterile protective blister packaging 110, showing two cups 50.1, 50.2 having different sizes, cups having conical form, for example forming a circularly-shaped funnel with different diameters forming a collection cavity with different collection volumes, syringe 20 is shown to be filled with a solution and container 80 is shown with a seal 82, for example a sealing cap, and two different insertion cannulas 30.1, 30.2 are shown with different diameters of the conical cup elements 50.1, 50.2, designed to be suitable for different patient anatomy, according to the second aspect of the present invention.

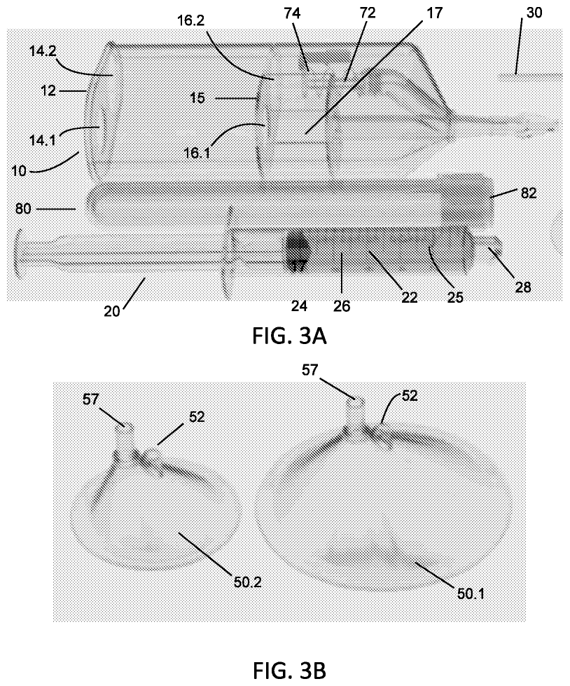

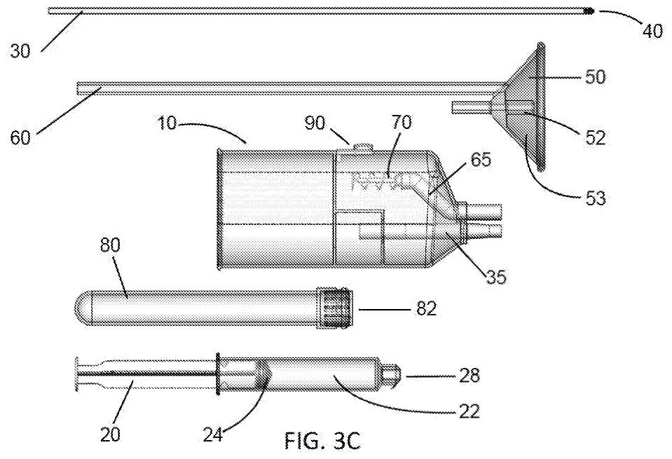

FIG. 3A shows a side perspective close-up view of the body 10, the container 80, and the syringe 20 of the kit to assemble a disposable uterine lavage device 100, body 10 made of a transparent material, body 10 having a side wall 12 at the distal end with two openings 14.1 and 14.2 for inserting syringe 20 and container 80, respectively, and having a traversing wall 15 substantially in the middle section of body 10 with two openings 16.1, 16.2 for laterally holding container 80 and syringe 20, the end at the spraying tip 40 being defined as the proximal end.

FIG. 3B showing the two cups 50.1, 50.2 of the kit, each cup having a conical structure, for example a funnel structure with a tunnel 52 or tube arranged in the center thereof for accommodating the insertion cannula 30, and having a tunnel or tube 57 for accommodating the extraction cannula 60 that is arranged offset but in parallel to the tunnel 52, when viewed along their axis of longitudinal extension, according to another aspect of the present invention.

FIG. 3C shows a side view of the elements of kit 200 where cup 50 has been placed over extraction cannula 60.

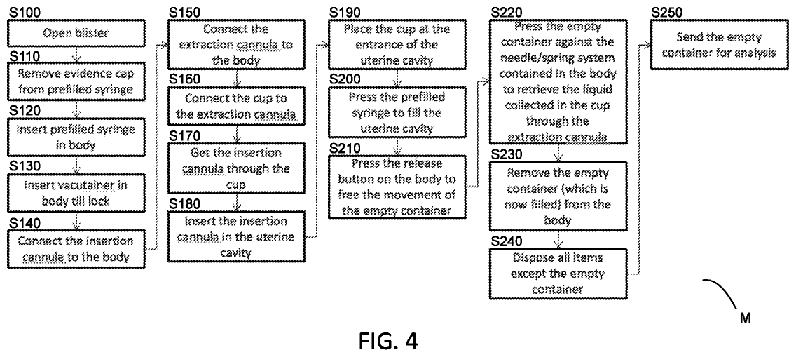

FIG. 4 shows schematic flowchart describing several steps of a method M of assembling and using disposable uterine lavage device 100 after a kit for the disposable uterine lavage device 100 has been delivered in a blister packaging 110 at a place of use, for example at the medical facility of a gynecologist or general practitioner, with steps S100 to S250, according to still another aspect of the present invention.

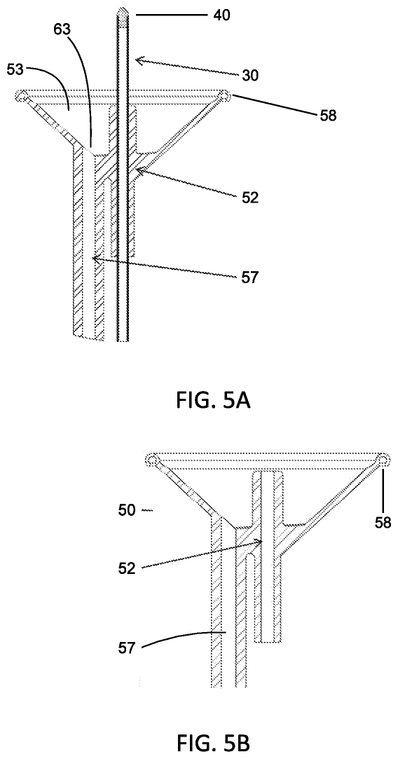

FIGS. 5A and 5B show a side cross-sectional views of cup 50, with FIG. 5A showing insertion cannula 30 inserted through tunnel 52 and having a dispensing or spraying tip 40 at the proximal end, and with FIG. 5B showing the cup with insertion cannula 30 removed. An entrance opening 63 for collecting a liquid solution to the extraction cannula 60 from the funnel 53 is arranged at the lowest point of a collection receptacle formed by the funnel.

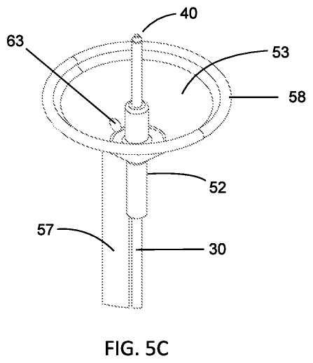

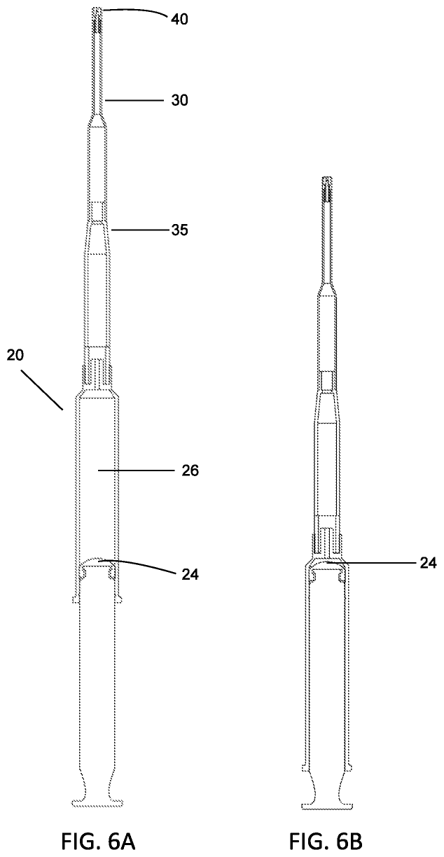

FIGS. 6A and 6B show a side cross-sectional view of syringe 20, fluidic pathway 35, insertion cannula 30 and spraying tip 40, for illustration purposes shown without the body 10, according to another aspect of the present invention, with FIG. 6A showing syringe 20 in an expanded or relaxed state, and FIG. 6B showing syringe 20 in a compressed state, to be used with disposable uterine lavage device 100, where a spraying of the solution up to the uterus roof is possible even when the user or patient is standing, being the level above the level of the fallopian tube entrances. The pressing of plunger 24 of syringe 20 can generate an aerosol that can make a foam composed of ambient air and a pressurized saline solution in the cavity 26 of syringe 20.

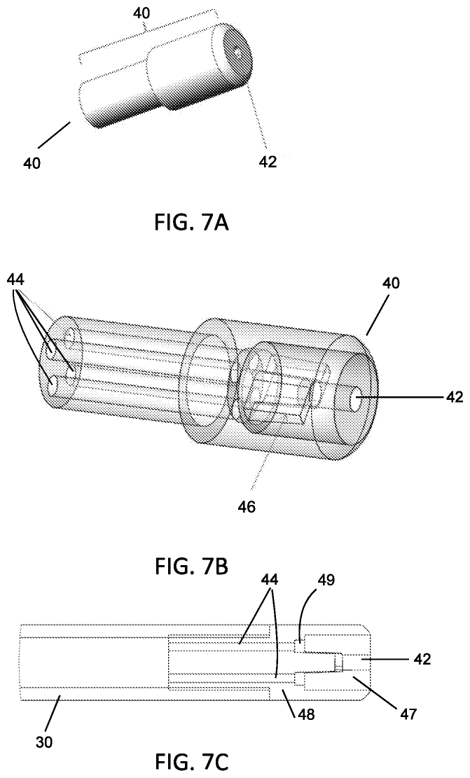

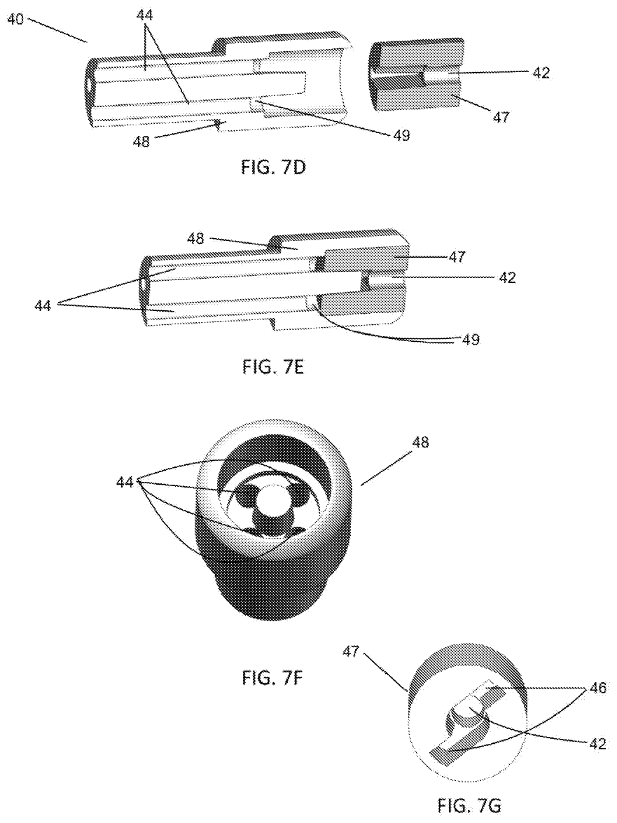

FIGS. 7A-7G show perspective views of different elements of the spraying or outlet tip 40 that can be attached to the proximal end of insertion cannula 30, with FIG. 7A showing spraying tip 40 with a spray outlet 42, FIG. 7B showing a semi-transparent view of spraying tip 40 showing a plurality of liquid insertion channels 44 on the cannula side, and one spray outlet 42 on the proximal side, with a chamber having flux separation blades 46 therebetween, FIG. 7C showing a cross-sectional view of the spraying tip 40 inserted into insertion cannula 30, showing two parallelly arranged insertion channels 44 arranged in parallel, FIG. 7D showing a perspective cut view of the two parts 47, 48 of spraying tip 40 separated from each other, FIG. 7E showing a cut view with the two parts inserted to in each other, and FIG. 7F showing a top perspective view of first part 48, and FIG. 7G showing a bottom perspective view of the second part 47.

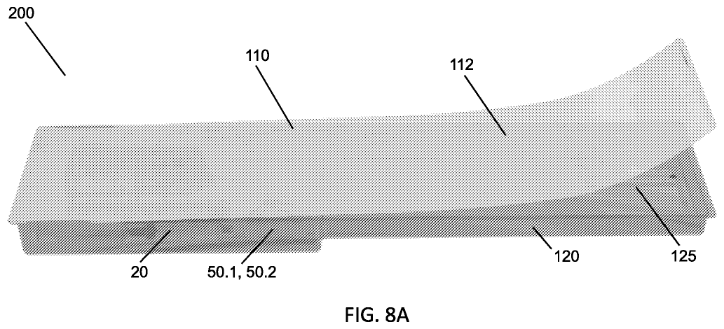

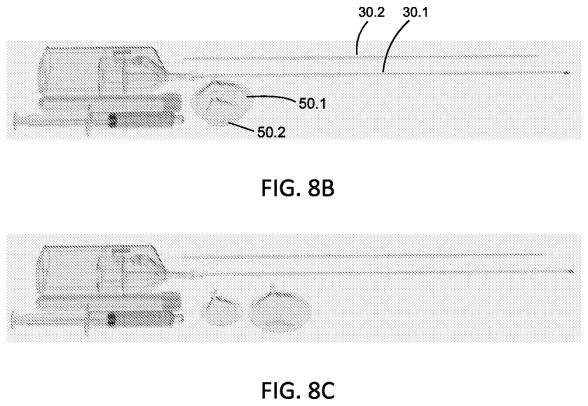

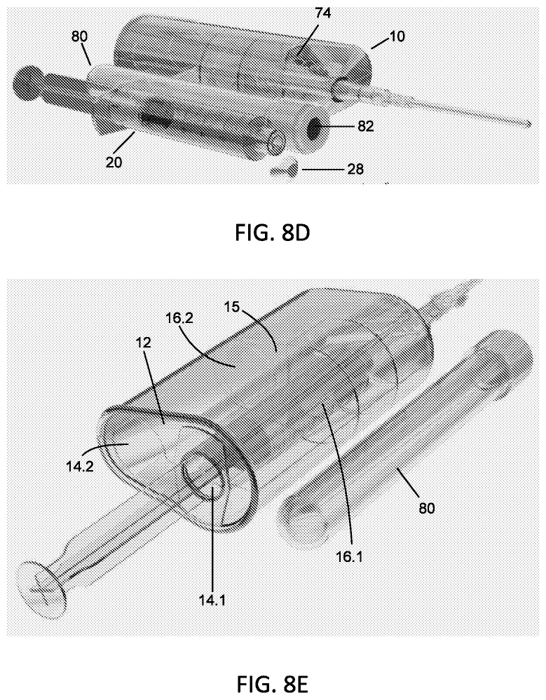

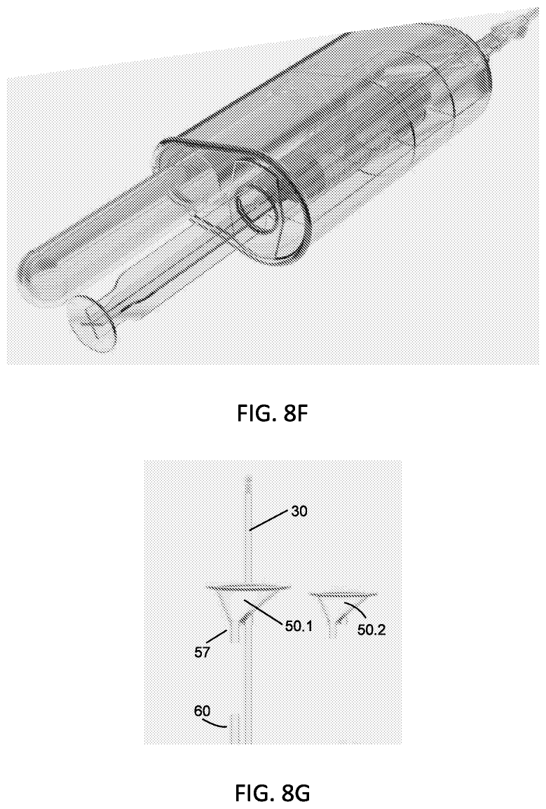

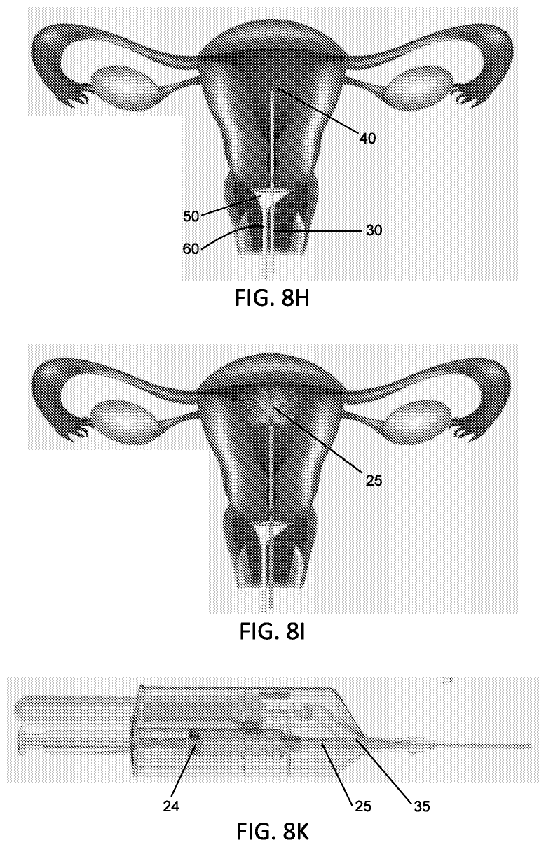

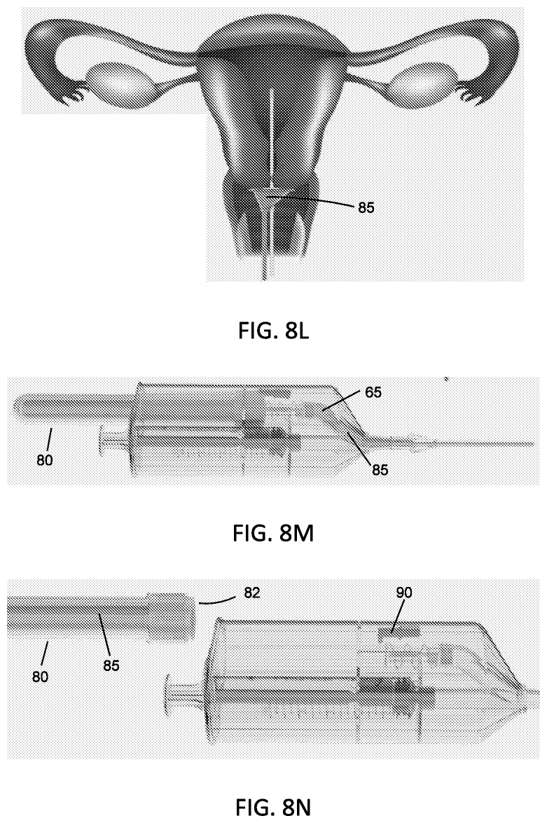

FIGS. 8A to 8O show side and perspective views of different stages in a method of unpacking, assembling, and using the disposable uterine lavage device 100, based on the flowchart shown in FIG. 4, according to an aspect of the present invention, with FIG. 8A showing the opening of the blister packaging 110 by removing the lid 112 (step S100), FIG. 8B showing the layout of the elements of the device 100 inside the blister packaging tray 120, with two differently-sized cups 50.1, 50.2 superposed over each other, FIG. 8C showing the larger cap 50.1 being removed from smaller cup 50.2, FIG. 8D showing the manual removal of cap or seal 28 from syringe 20 serving as a sealing means and tamper detection (step S110), FIG. 8E showing the insertion of syringe 20 to body 10 (step S120), FIG. 8F showing the insertion of container 80 to body 10 (step S130), FIG. 8G showing a side view where the cup 50 is inserted to connect to both insertion cannula 30 and extraction cannula 60 (steps S160, S170), FIG. 8H showing the insertion of insertion cannula 30 into the endometrial or uterine cavity of the uterus (step S180), with cup 50 located at the entrance to of the cervix and the uterine cavity (step S190), FIGS. 8I and 8K show the dispensing of the solution 25 inside the endometrial cavity (FIG. 8I) by activation of the plunger 24 of syringe 20 (FIG. 8K) (step S200), FIGS. 8L and 8M show the collection and retrieval of the solution 85 from outside the cervix (FIG. 8L) and the collection of the solution in the container 80 (FIG. 8M) via cup 50 and extraction cannula 60 (step S210, S220), the cup 50 serving as a collector element, FIG. 8N showing the removal of the container 80 from the body 10 (step S230) and the disposal of all elements other than the container 80 (step S240), and FIG. 8O showing the container 80 being marked with a label 87 or other type of recording for the specific patient, before sending to another entity for examination and testing (step S250).

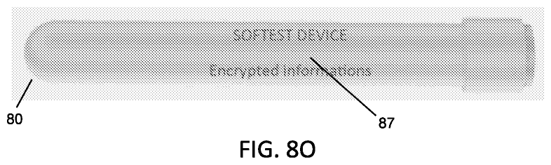

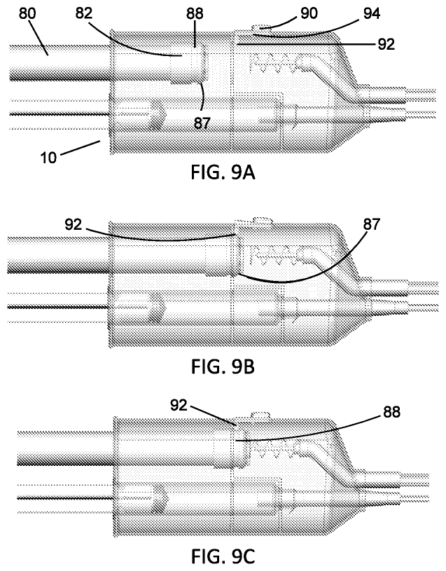

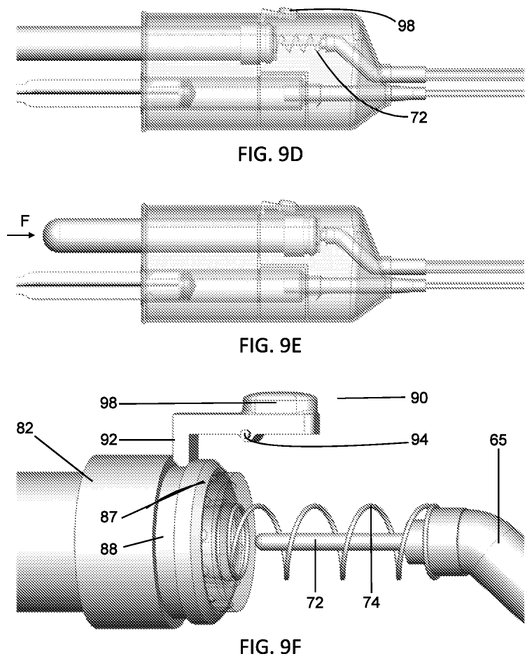

FIGS. 9A to 9F showing the steps of operation of the safety mechanism 90, safety mechanism 90 configured to block the user or operator for accidentally pushing container 80 into operative engagement with needle and spring mechanism 80, and requiring a manual release by button or handle 98, as shown with steps S210 and S220.

FIG. 1A shows an assembled disposable uterine lavage device 100, and FIG. 1B shows a perspective of the assembled device, according to a first aspect of the present invention. Device 100 includes a body 10 for holding several elements of the device 100 together and for allowing a user to hold the device 100 for operation, in an embodiment with a single hand. Body 10 includes a distal wall end 12 having two openings 14, one opening 14.1 for inserting a syringe 20, and one opening 14.2 for inserting a container 80. To facilitate the operation and to have visual feedback of the dispensing and collection of the solution in at least one embodiment, body 10 is made of a transparent material. Next, device 100 includes a prefilled syringe 20 for containing and spraying or injecting a solution into the uterine cavity, syringe 20 can be inserted for operation to in body 10, and its dispensing tip at the proximal end can be covered by a removable seal 28. In at least one embodiment, the solution is a saline solution. A fluidic pathway 35 fluidically connects the chamber 26 of syringe 20 with the insertion cannula 30, the insertion cannula removably attachable to the fluidic pathway 35. This is done via a cylindrical structure 17 of body that accommodates front part of the syringe 20 upon insertion to body 10. The inner dimensions of cylinder 17 can be such that front part of syringe 20 forms a press-fitted engagement with cylinder, forming a liquid-tight seal. At the proximal end of the insertion cannula 30, a spraying tip 40 is arranged, while the distal end of cannula 30 is configured to tightly engage with fluidic pathway 35 of body 10. Fluidic pathway 35 can be made as a part that is fixedly installed or an integral part of the body 10, and has a proximal end that allows for insertion of insertion cannula. In FIG. 2B, two different insertion cannulas 30.1 and 30.2 are shown, one already inserted to the proximal end of the fluidic pathway 35.

Fluidic pathway 35 and insertion cannula 30 are configured to guide the solution 25 ejected from cavity 26 of syringe 20 through body 10, towards the spraying tip 40 for spraying and dispensing the solution 25 inside the uterus, once insertion cannula 30 is inserted (see FIGS. 8H and 8I). Next, a cup 50 is arranged to be removably placed over both insertion cannula 30 for the dispensing and extraction cannula 60 for the collecting the lavage solution released by the uterus by gravity (see FIG. 8G). Cup 50 is formed to be made of a very flexible material for insertion into the vagina below the cervix, for example a medical grade silicon or other type of soft biocompatible synthetic material. An extraction cannula 60 is configured to collect the solution 85 from cup 50 to guide it towards body 10, a needle and spring system 70 for engaging with a container 80, and a safety mechanism 90 for allowing the needle 72 of needle and spring system 70 to pierce or otherwise penetrate a seal 82 of container 80. Needle 72 is hollow and provides for a fluidic path between container 80 and fluidic pathway 65 when the seal 82 is pierced.

Container 80 can be inserted to body 10 via opening 14.2 and 16.2 without that the container is opened to preserve a depressurization therein, for example without that the seal 82 is pierced or broken. Thereafter, it is possible to push container 80 further towards body 10, for example with a thumb or other finger of operator, such that needle 72 traverses and pierces seal 82 of container 80. Thereby, spring 74 urges against container 80, so that a predetermined force is required to push container 80 towards needle 72 for piercing. A fluidic pathway 65 fluidically connects collection opening 63 of extraction cannula 60 to container 80 by a termination with needle 72 at the end of the fluidic pathway 65 that faces container 80. If container 80 is under a certain level of vacuum or depressurization, for example the pressure inside container 80 is lower than the ambient pressure, the piercing of seal 82 will allow to aspire a solution via hollow needle 72, fluidic pathway 65, and via collection opening 63 from cup 50. Container 80 can be removably locked to body 10 by different mechanisms, for example safety mechanism 90 allows to snap-in or otherwise to lock the engaged container 80, so that container 80 can only be removed from body 10 by the user disengaging the safety mechanism from container 80. Also, in another variant, safety mechanism 90 can block the pressing of container 80 towards needle 72, to avoid that the container 80 is inadvertently pierced by needle 72. Upon removal of container 80 from body 10, seal 82 is configured to hermetically close container 80 when needle 72 is pulled out or retreated, such that container 80 can be sent to a different entity for further processing whilst preserving the liquid 85 therein.

In the variant shown, safety mechanism 90 includes a clip 92 that can engage with a ledge formed by the cap or seal 82 of container 80, or the clip 92 can engage with a groove 88 of rear wall of container 80 to block the manual pressing of container 80 farther into body 10 to prevent piercing by needle 72, and can be disengaged by the user pulling mechanism towards the proximal end (spray tip end 40). In another variant, once container 80 is inserted to body 10 but the seal is not yet pierced, it can be turned in either clockwise or counterclockwise direction to snap-in by a bayonet mechanism or mount, to avoid that container 80 falls out from body 10.

Body 10 is shown to be made of transparent material such that a scale 22 of syringe can be seen by the user through the transparent walls of body 10, to show the interior of body 10, and allows of a visible inspection of a connection between needle 72 and container 80. Also, the elements forming the fluidic connections, including fluidic pathways 35 and 65, can be made of transparent material, as shown in FIG. 2A, to allow a user to visually inspect a state of the dispersion and the collection of solutions 25, 85. Body 10, along with syringe 20 and container 80 can be made of polymeric material, possibly made of polylactide (PLA) or Copolyester materials to limit the carbon footprint of the product lifecycle and facilitate the recycling of the disposable parts.

The device 100 as shown in FIGS. 1A and 1B allows for a one-hand and one-time use when performing the uterine lavage. For example, body 10 can be dimensioned such that it can be held by one hand, while the thumb of the same hand is still able to press either syringe 20 to inject solution 25 or press container 80 to collect solution 85 by depressurizing the fluidic path from cup 50 via extraction cannula 60 and fluidic pathway 65 towards funnel 53, acting as a suction container. In at least one embodiment, for one-hand operation, body 10 is less than 8 centimeters wide. Blocking mechanism 90 can be released by a finger other than the thumb, for example by the index finger or middle finger. Through the transparent walls, the operator can view a scale 22 on syringe 20 to provide for the required doses of solution 25.

As shown in FIGS. 2A and 8A and 8B, according to another aspect of the present invention, disposable uterine lavage device 100 can be delivered as a single use kit 200 that is located in a sterile packaging, for example a blister packaging 110 with a blister tray 120 with pockets 125 for the individual parts or elements that can be assembled to form the disposable uterine lavage device 100. Blister packaging 110 of kit 200 allows to safely store kit 200 before use, and allows delivery of kit 200 in a sterile state. The kit 200 and device 100 can allow a single or one-time disposable use for washing the uterus quickly and collecting the solution egressed from the uterine cavity. Solution 25, for example but not limited to a saline solution 25, is thereby already filled into cavity 26 of syringe 20, and the front proximal dispending end of syringe is sealed and protected by cap or seal 28. Once the dispensing and collection of solution has occurred, it is possible to discard most elements of kit 200 and device 100, other than container 80 that collected the solution 85. The container can be vacutainer or a container having a seal 82 with a centrally located membrane that is pierceable by needle 72 of the needle and spring system 70 mechanism.

In at least one embodiment, syringe 20 is delivered with kit 200 prefilled with solution 25, in another embodiment between 2 and 10 cubic centimeters (cc) of saline solution. Syringe 20 is delivered in a hermetically sealed state, having an appropriate plunger 24 for his its purpose, and having a seal or cap 28 on the front tip, for example an evidence cap that shows if it has been tampered with. Also, in at least one embodiment the container 80 has a volume capacity in the range of 5 to 15 cc, in another embodiment around 10 cc, to able to collect all of solution 85 received via cup 50.

FIGS. 3A, 3B, 3C, 5A and 5B, show the two different cups 50.1 and 50.2 of the kit 200, that are configured to engage with body 10, used to collect the solution 85 that is egressed from uterine cavity by gravity, according to another aspect of the present invention. Regardless of the different diameters and also different volume capacities of the conical element of the cup 50, i.e., the funnel 53, both cups 50.1 and 50.2 have two conduits arranged in parallel to each other, and also arranged at the same distance to each other, having the same dimensions for both cups 50.1, 50.2, for attachment to both fluidic pathways 35, 65 of the cups 50.1, 50.2, respectively. This allows to attach different sized cups 50.1, 50.2 in terms of volume to the same body 10. In the variant shown, the kit 200 has two different sized cups 50.1, 50.2, but it is also possible that there are more than two different cups. This allows the operator to choose a specific cup based on different anatomy of the patient. Also, an upper circular edge 58 of conical part or funnel 53 is rounded, for example has a tubular structure to form a circular toroid shape, and is made from a soft elastic material that allows for easy manual compression for insertion and ensure soft and harmless contact with the cervix. For example, the material for conical part 53, in particular the circular edge 58 can be made to be much softer that the material for tunnel 52 and extraction cannula 60.

For example, as shown in FIG. 3C, the portions of fluidic pathways 35, 65 that protrude outside of body 10 are arranged in parallel to teach other, with an opening that allows to accommodate insertion cannula 30 and extraction cannula 60, respectively, for example by a press-fitted engagement. Also, a distance between the protruding elements of fluidic pathways 35, 65, measured from a center thereof, is the same as the distance between tunnel 52 and tunnel 57 of cup 50, measured from a center thereof. This allows inserting insertion cannula 30 and extraction cannula 60 to both body 10 and cup 50 such that the cannulas 30, 50 remain parallel to each other. In a variant, it is possible that extraction cannula 60 and tunnel or tube 57 are the same integrated element, as being part of the cup 50, for example as shown in FIG. 1E and 3C.

Similarly, as shown in FIG. 2B, kit 200 is shown with two different insertion cannulas 30.1, 30.2 having different lengths, but there could be more than two insertion cannulas with different lengths. Each cup 50.1, 50.2 has a first conduit that forms part of the extraction cannula 60, that can be connected to a connection end of fluidic conduit 65 of body 10. Also, at the lowest level of funnel 53, a collection opening 63 is formed for extraction cannula 60 for collecting solution 85 that accumulates in funnel 53. Also each cup 50.1, 50.2 has a conduit in the form of a tunnel 52 that is centrally arranged with the funnel 53, configured to engage and hold the insertion cannula or tube 30. FIG. 5A shows the insertion cannula 30 engaged with tunnel 52, and FIG. 5B shows cannula 30 removed from tunnel 52. With this design of cups 50.1, 50.2, and the two parallel arranged conduits 52, 60, as tunnel and extraction cannula, different sized cups can be connected to body 10 for injection and extraction of solutions 25, 85.

According to another aspect of the present invention, a method M of using kit 200 is provided, in at least one embodiment in a one-time use operation. FIG. 4 shows schematic flowchart describing several steps S100 to S250 of method M of receiving kit 200 at a place of use, unpacking the kit 200, assembling the kit 100 to be a useable but disposable uterine lavage device 100, using disposable uterine lavage device 100 for extracting solution 85, discarding all elements but for the container 80 filled with solution 85, and preparing and sending container 80 for further analysis to a different locations. Moreover, FIGS. 8A to 8O show different stages of method M.

First, sterile kit 200 has been delivered in a blister packaging 110 at a place of use, for example but not limited to at the medical facility, medical examination room, medical office, primary care exam room, clinical space of a gynecologist, hospital, care center. In step S100 of method M, at the place of use, the operator, for example but not limited to gynecologist, nurse, technician, doctor, physician, general practitioner, shortly before the examination, unpackages unpacks and opens the packaging 110 to peel off or otherwise remove lid or cover 112, and takes out the tray 120 from packaging 110 that has all the elements of device 100, as shown in FIG. 8A. FIG. 8B shows the layout of the elements of the device 100 inside the blister packaging tray 120, with two differently-sized cups 50.1, 50.2 superposed over each other, FIG. 8C showing the larger cap 50.1 being removed or lifted from smaller cup 50.2 that can also be part of step S100. Also, syringe 20 is delivered with cavity 26 already pre-filled with a solution 25. In step S110 and as shown in FIG. 8D, the operator manually removes of cap or seal 28 from pre-filled syringe 20 having a solution 25 in cavity, serving as a sealing means but also allowing operated to see if the syringe has been tampered with.

In a variant, instead of having a removable cap 28, syringe 20 can be sealed by a membrane, for example an aluminum foil that is bonded to the tip of syringe 20, and the membrane is not removed by the user or operator. Instead, the membrane can be pierced by a hollow needle that is in fluidic connection with the fluidic pathway 35, similar to needle 72 that pierces seal 82 of container 80. This allows user to press syringe 20 into body for fluidic connection with fluidic pathway 35. A fixation mechanism can be used so that syringe is not easily removed from the connected position, to preserve a liquid-tight seal between syringe 20 and fluidic pathway 35, for example but not limited to a press-fitted engagement between the tip of syringe 20 and cylindrical structure 17, by a bayonet mechanism, clip-in mechanism.

Next, in step S120, as shown in FIG. 8E, the operator inserts syringe 20 to body 10 via opening 14.1 so that tip of syringe firmly engages with cylinder 17, and in step S130, shown in FIG. 8F, the operator inserts container 80 to body 10 via opening 14.2 and 16.2 such that safety mechanism 90 engages with container 80 to hold the container at a fixed place in body 10. Next, in a step S140, the insertion cannula 30 is inserted to safely engage fluidic pathway 35 of body 10. In this step, the operator can choose between insertion cannulas 30.1, 30.2 having different lengths. Thereafter, in a step S150, the extraction cannula 60 is attached to fluidic pathway 65 of body 10. Next, in steps S160 and S170, one of the silicon cups 50.1, 50.2 is chosen and attached to both insertion and extraction cannulas 30, 60, as shown in FIG. 8G depicting a side view where the cup 50 is inserted to connect to both insertion cannula 30 and extraction cannula 60. The external radial diameter of cups 50 can be in a range between 30 mm and 50 mm, in at least one embodiment between 40 mm to 46 mm, for example four (4) cups 50 with the diameters 40 mm, 42 mm, 44 mm, and 46 mm. In case the extraction cannula 60 is an integral part of cup 50 with cannula 60 and tunnel 57 a combined element, step S160 need not be performed. Steps S110, S120, S130, S140, S150, S160, and S170 form part of a step of assembling the kit 200 to form device 100. These steps do not have to be necessarily performed in this order. For example, insertion and extraction cannulas 30, 60 can be attached to cup 50 in steps S160, S170 before the cannulas 30, 60 are attached to body 10 in steps S140, S150. Also step S120 can be performed after cannulas 30, 60 are attached to body 10.

Thereafter, a step S180 is performed as shown in FIG. 8G where the operator inserts insertion cannula 30 into the endometrial or uterine cavity of the uterus, and a step S190 is performed where cup 50 is entered to the vagina at the entrance to the cervix and uterine cavity. Next, a step S200 is performed where the operator presses or contracts syringe 20 to dispense the pre-filled solution 25 to fill the uterine cavity. FIGS. 8I and 8K are part of step S200 and show the dispensing of the solution 25 inside the endometrial cavity (FIG. 8I) by activation of the plunger 24 of syringe 20 (FIG. 8K). It can be seen that solution 25 exits by spraying tip 40 and touches the upper wall of the uterine cavity.

Solution 85 that was present in uterine cavity passes cervix by gravity and can collect in funnel 53 of cup 50, shown in FIG. 8L.

Step S200 allows the operator to precisely control a pressure of solution 25 that is delivered to uterine cavity of the patient. An increase of the overall pressure inside uterine cavity will be felt as an increase of resistance to the motion of plunger 24 of syringe 20.

This force feedback allows the operator to control a quantity of solution 25 is delivered to uterine cavity 25. For example, it is desirable that no solution 25 enters the uterine fallopian tubes when solution 25 is filled or injected to the uterine cavity. This can be detected by the operator who is pushing syringe 20 with a finger upon feeling a sudden increase in press resistance, for example his thumb. With the device 100 as described herein, the lavage of the uterine cavity is strongly simplified, and performed by a one-hand operation of operator. The only operating that requires some feedback control by the skill of the operator is the pressing or pushing of the sole syringe 20 that delivers solution 25 to the uterine cavity.

Next, a step S210, container 80 is pressed into body 10 to permit solution 85 or fluid that has accumulated inside funnel 53 to pass via cup 50, extraction cannula 60, fluidic pathway 65, and needle 72 to reach an interior of container 80 by suction due to the vacuum or depressurization of the interior of container 80. Moreover, the fluid flow from cup 50 to container 80 can be further supported by gravity. This can be done by a step S200 performed by operator urging container 80 towards needle 72 against the biasing force of spring 74 via opening 14.2 to press container 80, (for example a vacutainer,) to overcome the force of spring 74 so that needle 72 will pierce and break seal 82. In another variant, a button can be pressed on body 10, being part of the safety mechanism 90, which releases needle 72 to pierce seal 82, the needle 72 being pushed by the release of a pre-tensioned spring 74.

Next, in step S230, solution 85 fills into container, by the fluidic path along cup 50, extraction cannula 60, fluidic pathway 65, and needle 72 to reach an interior of container 80. This is shown exemplarily in FIGS. 8L and 8M depicting the collection and retrieval of solution 85 from outside the cervix (FIG. 8L) and the collection of the solution in the container 80 (FIG. 8M) via cup 50 and extraction cannula 60, the cup 50 serving as a collection volume. During this step, the patient can be in a standing position or upright position, so that the solution 82 is better collected by cup 50. As explained above, container 80 is delivered to have a vacuum therein or at least a partial depressurization to have a lower pressure therein than the ambient air, and upon piercing of the seal 82, in addition to the force of gravity on solution 85, container 80 aspires solution 85. Next, in a step S230, container 80 filled with solution 85 is removed from body 10, and optionally the aspiration is finished, as shown in FIG. 8N. Thereafter, in a steep S240, as the elements of the kit 200 are disposable, all elements other than the container 80 are discarded and not used again, and in step S250, the container 80 is marked with a label 87 or other type of recording or identification tag for the specific patient, to provide for patient-identification information to container 80, and to provide for additional data related to the solution collection, before sending to another entity for examination and testing. Specifically, solution 85 can be used for detecting endometrial or ovarian cancer. For example, calls are collected from solution 85, and cane be analyzed from container 80 by centrifugation to detect for the presence of somatic mutations in the gene TP53 and other selected genes by ultra-deep, targeted sequencing.

According to another aspect of the present invention, a specific spraying tip 40 and syringe 20 is provided that can operated together with kit 200 or device 100, as shown in FIGS. 6A, 6B, 7A, 7B, 7C, 7D, 7E, 7F, and 7G. FIGS. 6A and 6B shows side cross-sectional views of syringe 20 in an expanded or relaxed state (FIG. 6A), and in a compressed state with all or most of solution 25 pressed out of syringe cavity 26 by plunger 24 (FIG. 6B). In combination with the spraying tip 40 of FIGS. 7A-7G, a spraying of the solution 25 up to the uterus roof is possible even when the user or patient is standing, being the level above the level of the fallopian tube entrances. For example, spraying tip 40 is configured such that it can spray the solution 25 in the form of an aerosol by an angle covering 180° with a main or central direction towards the upper wall of the uterine cavity, along the axis of longitudinal extension of insertion cannula 30. The pressing of plunger 24 of syringe 20 generates a pressure on solution 25 that in turn can generate an aerosol that causes a foam inside the uterine cavity, composed of ambient air and a pressurized saline solution in the cavity 26 of syringe 20. FIGS. 7A, 7B and 7C show perspective views of the spraying or outlet tip 40 attached to the proximal end of insertion cannula 30, which in turn is fluidically connected to fluidic pathway 35 and syringe 20, with FIG. 7A showing spraying tip 40 with a spray outlet 42, FIG. 7B showing a semi-transparent view of spraying tip 40 showing a plurality of liquid insertion channels 44 on the cannula side, and one spray outlet 42 on the proximal side, with a flux separation chamber 46, and FIG. 7C showing a cross-sectional view of the spraying tip 40 inserted into insertion cannula 30, showing two parallelly-arranged insertion channels 44, in parallel to an axis of longitudinal extension of cannula 30.

The spraying tip 40 includes a vaporizer mechanism, in an embodiment located at the end of cannula 30, and is composed of two, in an embodiment made out of polymer, parts 48, 47. The pressure generated by syringe 20 that allows to spray solution 25, for example the washing solution, is guided through cannula 30 into the first part 48 of spraying tip 40, and passes through a plurality of parallelly-arranged insertion channels 44. Next, solution is accumulated under pressure in a circular chamber 49 at the interface with the second part 47 of spraying tip 40. The transition from the conduit formed by insertion cannula 30 having a relatively small cross-sectional area, to the plurality of parallelly-arranged insertion channels 44 and thereafter the circular chamber 49, having a larger overall cross-sectional area as compared to cannula 30, depressurizes solution 25, such that a foggy dispersion of solution 25 is created having a large number of small droplets. It is possible that the two parts 48, 47 are attached to each other by an adhesive.

Next, solution 25 is then passed into an opening that forms two or more radially extending flux separation chambers 46, and then exits under pressure via a single exit nozzle or spray outlet 42, in the second part 47. The created droplets from solution 25 are further dispersed by flux separation chambers 46, and the exit nozzle or spray outlet 42 then provides for a means to spray dispersed solution 25 forming an aerosol from outlet 42. Upon exit of droplets from nozzle 42, ambient air is can be mixed with the exiting droplets to form an aerosol. In the variant shown, spraying tip 40 includes a first and second part 47, 48 that be pressed into each other with a press-fitted engagement, to form a liquid-tight seal between these two elements. Upon exit of aerosol made from solution from tip 40 into uterine cavity by exiting nozzle 42, a foam is generated by mixing ambient air and a saline solution that is generated by a pressure from plunger 24 of syringe 20. The aerosol is sprayed into the uterine cavity of the patient, to reach all or a substantial part of the internal tissues of the uterus. The spraying angle covers about 180° of a half-sphere with a main rotational axis coinciding with an axis of longitudinal extension of cannula 30.

The arrangement of the spraying tip 40 at the end of insertion cannula 30 provides for certain advantages. By preserving the pressure generated by syringe 20 all the way to the spraying tip 40 when plunger 24 is being pushed in, a better filling and distribution of the aerosol in the uterine cavity is achieved, ensuring that the entire cavity is probed, leading to a decrease of false-negative detections. It also provides for a higher pressure of the injected solution, enhancing the cells of the uterine cavity, increasing the detection strength also leading to a decrease of false-negative detections, as it allows capturing uterine cells at the top wall of the uterus in the entire uterine cavity. In an embodiment, the cannula 30 is made of bio-compatible materials, in at least one embodiment of plastic materials, in a still other embodiment of medical-grade silicone. Moreover, cannula 30 can have an external diameter in the range of 5 mm to 2 mm, in at least one embodiment 3 mm, and can have an internal diameter as a fluid conduit in the range of 3 mm to 1 mm, in an embodiment 1.5 mm.

FIGS. 9A to 9F showing the steps of operation of the safety mechanism 90, safety mechanism 90 configured to block the user or operator for pushing container 80 into operative engagement with needle and spring mechanism 80, and requiring a manual release by button or handle 98, as shown with steps S210 and S220. In the variant shown, as shown in FIG. 9F, safety mechanism 90 includes a lever-like element that includes a pivot 94, a clip 92 for engaging with seal 82 of container 80, a press button 98 that protrudes out of body 10 via an opening, and the seal 82 includes a circular groove 88 surrounding seal 82, and a beveled edge 87.

In FIG. 9A, the insertion of container 80 to body 10 is shown, via opening 14.2. Syringe 20 is already inserted. As shown in FIG. 9B, the beveled edge 87 of seal 82 of container 80 abuts against clip 92, and urges clip 92 upwards by a rotation around pivot 94. Next, as shown in FIG. 9C, clip 92 engages with groove 88 to hold container 80 in a first, idle position relative to body 10. Next, as shown in FIG. 9D, user or operator presses button 98 inside body 10, so that clip disengages from groove 88, and then presses with force F to push container 80 against spring 92, as shown in FIG. 9E. This brings container 80 into a second position relative to body 10, where the seal 82 is pierced and broken, such that the under-pressure in empty container 80 can aspire liquid or solution 85 via hollow needle 72 and fluidic pathway 65.

While the invention has been disclosed with reference to certain preferred embodiments, numerous modifications, alterations, and changes to the described embodiments, and equivalents thereof, are possible without departing from the sphere and scope of the invention. Accordingly, it is intended that the invention not be limited to the described embodiments, and be given the broadest reasonable interpretation in accordance with the language of the appended claims.

According to one aspect of the present invention, a uterine lavage kit for collecting a solution from a uterine cavity of a subject. The uterine lavage kit includes a body, an insertion cannula, a syringe for injecting a solution via the insertion cannula into the uterine cavity, a collection cup for collecting the solution egressed from the uterine cavity after the injection, a removable container configured to be removed from the body, an extraction cannula in fluidic connection with the collection cup, the extraction cannula configured to guide the solution from the collection cup towards the removable container.

According to another aspect of the present invention, a method for performing uterine lavage for detecting endometrial or ovarian cancer in a patient is provided. In at least one embodiment, the method includes the steps of receiving a single use uterine lavage kit in a sterile packaging, the single use uterine lavage kit having a syringe having containing a solution or liquid, an insertion cannula, a collection cup, and a container, opening the sterile packaging and accessing the uterine lavage kit, inserting the insertion cannula to a the uterine cavity of the patient, injecting the solution liquid by the syringe via the insertion cannula to the uterine cavity, collecting the solution by gravitational force in the collection cup, aspiring the solution by vacuum to the container, and sending the container to an accredited entity for additional processing to determine the presence of endometrial or ovarian cancer in the patient.

According to still another aspect of the present invention, a plurality of collection cups for a uterine lavage kit for collecting a solution egressed from a uterine cavity of a subject, are provided. In at least one embodiment, each collection cup includes a cavity for collecting the solution, a first conduit for engaging with an insertion cannula of the uterine lavage kit, the first conduit traversing the cavity, and a second conduit for engaging with an extraction cannula of the uterine lavage kit, the second conduit arranged in parallel to the first conduit and traversing the cavity, wherein each collection cup has a different diameter, and is made of a soft material for harmless insertion to a vagina of a patient and cervix sealing.

With the system, device and method according to some aspects of the present invention permit to provide for an early detection of cancer, and such early detection can lead to significantly higher survival rates of woman that are affected by the early stage cancer.

Therefore, it is possible that thousands of lives scan be saved by the use of the present method and device, and can provide for a large reduction of healthcare and treatment costs associated with ovarian or endometrial cancers, to bring about a significant change the way this these diseases are diagnosed and subsequently treated in the future.

Also, with the present device, system, and method, it is possible to entirely avoid anesthesia for the testing procedure, as the elements that of the device or system are introduced to the uterine cavity and the vagina are very soft.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A uterine lavage kit for collecting a solution from a uterine cavity of a subject, comprising:
a body;
an insertion cannula;
a syringe for injecting a solution via the insertion cannula into the uterine cavity;
a collection cup for collecting the solution egressed from the uterine cavity after the injection;
a removable container configured to be removed from body; and
an extraction cannula in fluidic connection with the collection cup, the extraction cannula configured to guide the solution from the collection cup towards the removable container.

2. The uterine lavage kit according to claim 1, further comprising:
a spraying tip arranged at the end of the insertion cannula,
wherein the spraying tip is configured to generate an aerosol including ambient air and the solution inside the uterine cavity of the subject upon pressing the syringe.

3. The uterine lavage kit according to claim 2, wherein the spraying tip is configured to generate the aerosol including ambient air and the solution inside the uterine cavity of the subject upon pressing the syringe, such that substantially all of internal tissues of the uterine cavity are reached.

4. The uterine lavage kit according to claim 2, wherein the spraying tip includes two polymer elements, one of the polymer elements having a plurality of liquid insertion channels that are arranged in parallel with an axis of longitudinal extension of the insertion cannula, the other one of the polymer elements having a spray outlet arranged to spray the aerosol from the proximal tip of the insertion cannula.

5. The uterine lavage kit according to claim 2, wherein an outlet of the spraying tip is configured to disperse an aerosol in a 180° direction with a central direction being along an axis of longitudinal extension of the insertion cannula.

6. The uterine lavage kit according to claim 1, wherein the insertion cannula is made of a biocompatible material and has an external diameter that is smaller than 5 mm, and has an internal diameter that is smaller than 3 mm.

7. The uterine lavage kit according to claim 1, wherein the collection cup is removably connected to both the insertion cannula and the extraction cannula.

8. The uterine lavage kit according to claim 1, wherein body is dimensioned to be held and manipulated by with one hand.

9. The uterine lavage kit according to claim 1, wherein the body, the insertion cannula, the syringe, the collection cup, the removable container, and the extraction cannula are packaged in a sterile packaging, and
the syringe is prefilled with the solution and an internal cavity of the removable container is depressurized.

10. A plurality of collection cups for a uterine lavage kit for collecting a solution egressed from a uterine cavity of a subject, each collection cup comprising:
a cavity for collecting the solution;
a first conduit for engaging with an insertion cannula of the uterine lavage kit, the first conduit traversing the cavity; and
a second conduit for engaging with an extraction cannula of the uterine lavage kit, the second conduit arranged in parallel to the first conduit and traversing the cavity,
wherein each collection cup has a different diameter, and is made of a soft material for insertion to a vagina of a patient.

11. The plurality of collection cups according to claim 10, wherein the cavity includes a circular hollow soft termination ring.

12. The plurality of collection cups according to claim 10, wherein the collection cups are removably connectable to the insertion cannula and the extraction cannula of the uterine lavage kit.

13. A method for performing uterine lavage for detecting endometrial or ovarian cancer in a patient, the method comprising the steps of:
receiving a single use uterine lavage kit in a sterile packaging, the single use uterine lavage kit having a syringe with a solution therein, an insertion cannula, a collection cup, and a container and an extraction cannula;
opening the sterile packaging and accessing the uterine lavage kit;
assembling the uterine lavage kit;
inserting the insertion cannula to a uterine cavity of the patient;
injecting the solution by the syringe via the insertion cannula to the uterine cavity;
collecting the solution by gravitational force in the collection cup; and
aspiring the solution by vacuum to the container through the extraction cannula.

14. The method for performing uterine lavage according to claim 13, wherein the step of aspiring includes a step of pressing the container into a body of the uterine lavage kit to pierce a seal of the container.

15. The method for performing uterine lavage according to claim 13, further comprising the step of:
discarding the syringe, the insertion cannula, the extraction cannula, and the collection cup after the step of aspiring.

16. The method for performing uterine lavage according to claim 13, further comprising the step of:
removing the container from the uterine lavage kit after the step of aspiring;
labeling the container with patient identification information; and
sending the container to an entity for additional processing to determine presence of endometrial or ovarian cancer in the patient.
